(19) United States
(12) Patent Application Publication
(10) Pub. No.: US 2017/0202379 A1
(43) Pub. Date: Jul. 20, 2017

(54) Smart Dishware with An Integrated Body Mass Index Reader

(71) Applicant: Nikola Vucicevic, Chicago, IL (US)

(72) Inventor: Nikola Vucicevic, Chicago, IL (US)

(21) Appl. No.: 15/410,517

(22) Filed: Jan. 19, 2017

Related U.S. Application Data
(60) Provisional application No. 62/280,262, filed on Jan. 19, 2016.

(51) Int. Cl. A47G 19/02; A61B 5/00; A61B 5/053; G01G 19/52; G01K 13/00

(52) U.S. Cl. CPC A47G 19/025; G01G 19/52; G01K 13/00; A61B 5/053; A61B 5/6887; A47G 2200/166; A47G 2200/226

(57) ABSTRACT

A dishware which allows a user to monitor his or her eating and health habits directly. The dishware includes an upper bowl-shaped body, a lower bowl-shaped body, a body mass index (BMI) reader, a microcontroller, and a housing base. The upper bowl-shaped body is detachable and washable. The BMI reader includes a first finger sensor and a second finger sensor. The first finger sensor and the second finger sensor are mounted to the lower bowl-shaped body. The lower bowl-shaped body is attached to the upper bowl-shaped body with a concave surface of the lower bowl-shaped body pressing against the convex surface of the upper bowl-shaped body. The housing base is centrally and adjacently connected to the lower bowl-shaped body, opposite the upper bowl-shaped body. The microcontroller is electronically connected to the BMI reader and internally mounted within the housing base.

The current application claims a priority to the U.S. Provisional Patent application Ser. No. 62/280,262 filed on Jan. 19, 2016.

### FIELD OF THE INVENTION

The present invention relates generally to dishware. More specifically, the present invention is dishware that is capable of reading and displaying various data relating to the food placed therein. The dishware also includes an integrated body mass index reader and heart shaped protrusion to raise awareness regarding the user's eating habits and physical health.

### BACKGROUND OF THE INVENTION

According to reports, it is estimated that 36 percent of adults and 17 percent of children and adolescents are considered to be obese. In other words, a third of Americans are considered to be unhealthy and overweight. Health risks of obesity include, but is not limited to, coronary heart disease, high blood pressure, increased chances of strokes, diabetes, various forms of cancer, reproductive problems and gallstones. Obesity is a growing epidemic with little to no signs of abating. A number of different solutions have been proposed to curb the epidemic, but have generally been unsuccessful due to the lack of psychological reinforcement needed to raise awareness. Therefore, the need for an apparatus that aims to provide psychological reinforcement, as well as a physical deterrent against poor eating habits is apparent.

The present invention is dishware that aims to facilitate in changing poor eating habits and therefore reduce obesity among its users. The present invention may be presented in the form of a bowl, plate or the like and comprises various components to raise awareness regarding the user's food, health and eating habits. The dishware comprises an integrated body mass index (BMI) reader, various sensors and wireless capabilities such that the user is able to monitor data regarding their food and health from a mobile device. Additionally, the present invention reduces the amount of food consumed through an integral protrusion so that the amount of food that the dishware can hold is limited. Therefore, the present invention reduces obesity in a variety of different ways.

### BRIEF DESCRIPTION OF THE DRAWINGS

FIG. 1 is a perspective view of the present invention.

FIG. 2 is an exploded perspective view of the present invention.

FIG. 3 is a front view of the present invention.

FIG. 4 is a sectional cut of the present invention in the exploded state taken about line A-A seen in FIG. 3.

FIG. 5 is an electronic diagram of the present invention.

FIG. 6 is an electric diagram of the present invention.

FIG. 7 is an electric diagram of an alternative embodiment of the present invention.

### DETAIL DESCRIPTIONS OF THE INVENTION

All illustrations of the drawings are for the purpose of describing selected versions of the present invention and are not intended to limit the scope of the present invention.

The present invention generally relates to dishware. More specifically, the present invention is an electronic bowl which collects data regarding a user's eating habits and body characteristics in order promote healthy eating habits. The present invention aims to reduce obesity through psychological reinforcement by providing a means for the user to check and view his or her body mass index (BMI) and body fat percentage prior to meal consumption. In doing so, the user is aware of where he or she currently stand regarding their BMI and body fat percentage and will likely be motivated to eat less, eat slower or eat healthier as such behavior will ultimately affect the displayed data. Furthermore, by checking one's BMI and body fat percentage during each meal, the user is able to see a trend and correct his or her behavior accordingly.

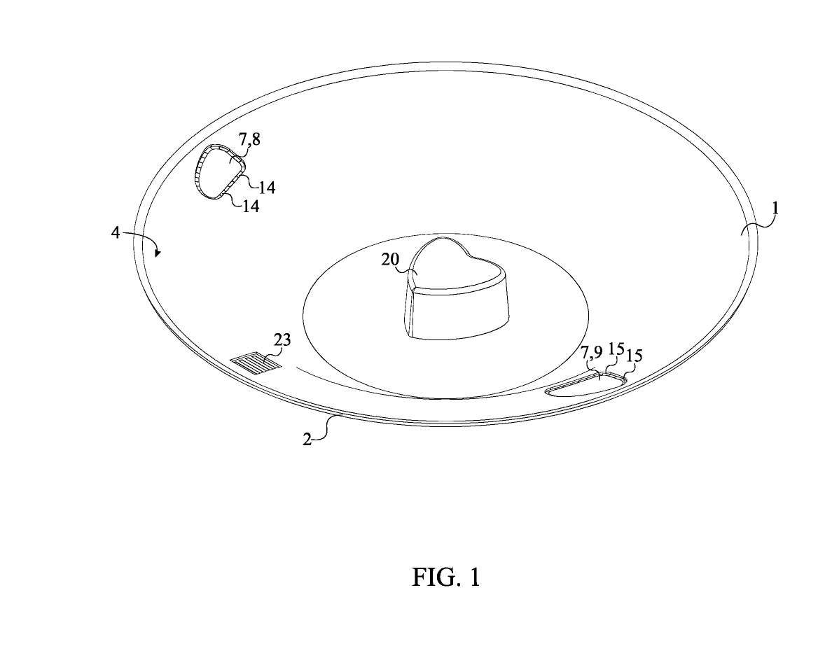

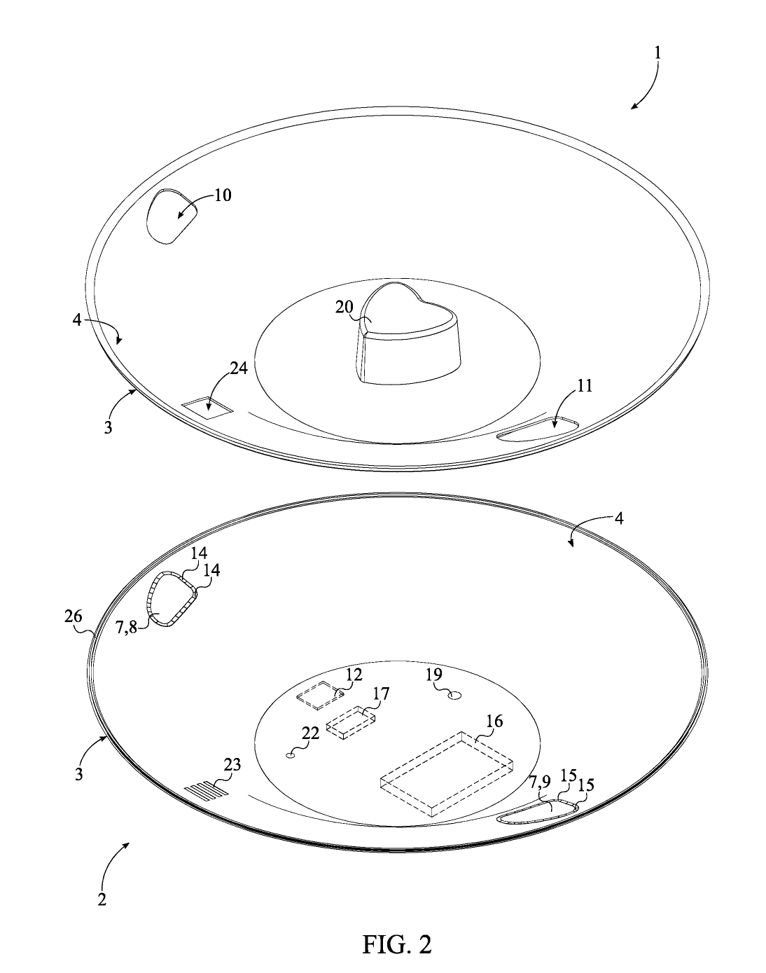

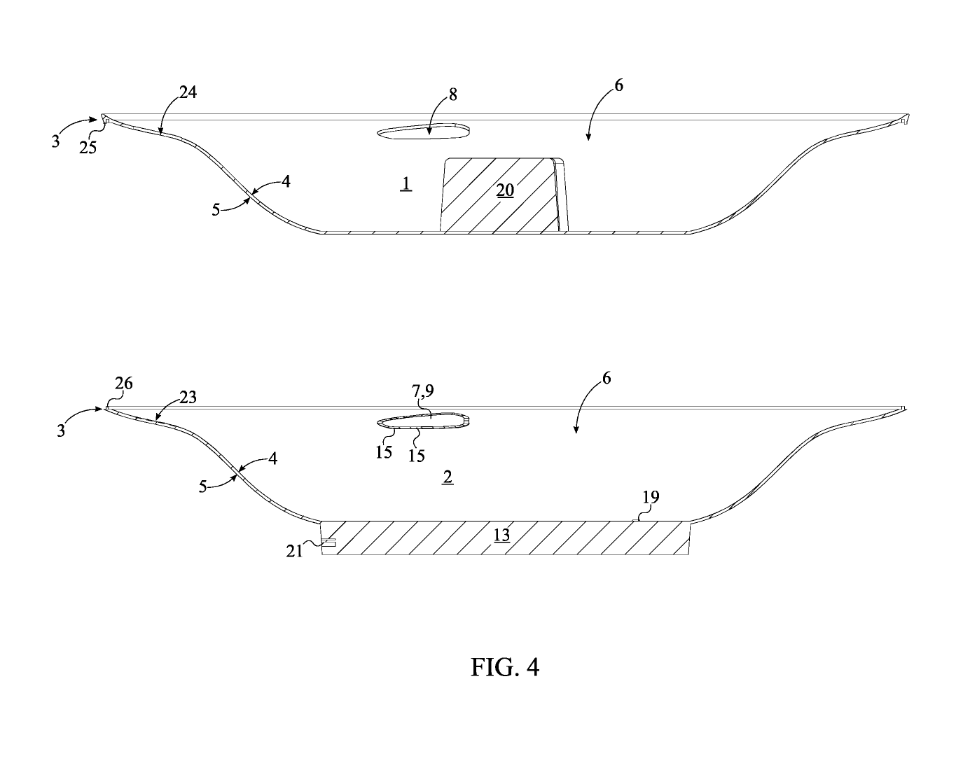

Referring to FIG. 1 and FIG. 2, the present invention comprises an upper bowl-shaped body 1, a lower bowl-shaped body 2, a BMI reader 7, a microcontroller 12, a housing base 13, a space-occupying protrusion 20, an at least one pressure sensor 19, a temperature sensor 22, a wireless communication device 17, an external computing device 18. The upper bowl-shaped body 1 is a hemispherical vessel such as a bowl, cup, plate, or any other dishware that is designed to hold food, solid and fluid. Thus, the upper bowl-shaped body 1 receives and holds food, and is preferably dishwasher safe. The lower bowl-shaped body 2 is shaped and sized complimentary to the upper bowl-shaped body 1 such that the upper bowl-shaped body 1 fully fits within the lower bowl-shaped body 2. The upper bowl-shaped body 1 is the detachable and washable half of the present invention. The lower bowl-shaped body 2 and the housing base 13 support the upper bowl-shaped body 1 and house the electronic components of the present invention. More specifically, the upper bowl-shaped body 1 and the lower bowl-shaped body 2 each comprise a concave surface 4 and a convex surface 5 as seen in FIG. 4. When the present invention is configured into a functional configuration, the lower bowl-shaped body 2 is attached to the upper bowl-shaped body 1. In particular, the concave surface 4 of the lower bowl-shaped body 2 is pressed against the convex surface 5 of the upper bowl-shaped body 1.

The BMI reader 7 measures the BMI of the user by sending an electrical signal from one portion of the user's body to another portion and timing the time it takes for said signal to travel through the user's body. In particular, the BMI reader 7 sends the signal from one hand of the user to another. This is accomplished through two points of contact with the user's body. Thus, the BMI reader 7 comprises a first finger sensor 8 and a second finger sensor 9. The first finger sensor 8 and the second finger sensor 9 are oval-shaped electrical conductors that are mounted to the lower bowl-shaped body 2. The user simply places a finger from one hand onto the first finger sensor 8 and a finger from the other hand onto the second finger sensor 9 for a predetermined amount of time. The first finger sensor 8 and the second finger sensor 9 may be positioned anywhere on the lower bowl-shaped body 2, although the optimal location is on either side of the lower bowl-shaped body 2. The housing base 13 is a cylindrical extrusion which vertically supports and stabilizes the present invention. The housing base 13 is positioned adjacent to the lower bowl-shaped body 2, opposite the upper bowl-shaped body 1. Additionally, the housing base 13 is centrally connected to the lower bowl-shaped body 2 to provide symmetric support to the upper bowl-shaped body 1 and the lower bowl-shaped body 2. The microcontroller 12 controls the BMI reader 7 and other electronics of the present invention in order to collect and manage gathered data. For example, the microcontroller 12 is electronically connected to the BMI reader 7 in order to send and time the electronic signal from the first finger sensor 8 to the second finger sensor 9. Then, the microcontroller 12 uses said measured time in conjunction with user's body information in order to calculate the user's BMI.

In the preferred embodiment of the present invention, the user is able to access the first finger sensor 8 and the second finger sensor 9 through the upper bowl-shaped body 1. This is achieved through a first finger-receiving cavity 10 and a second finger-receiving cavity 11. Referring to FIG. 2, the first finger sensor 8 and the second finger sensor 9 are positioned opposite to each other across a central cavity 6 of the lower bowl-shaped body 2. This positions the first finger sensor 8 and the second finger sensor 9 on either side of the present invention, one for each hand of the user. Additionally, the first finger sensor 8 and the second finger sensor 9 are each integrated into the concave surface 4 of the lower bowl-shaped body 2. In particular, it is preferred that the first finger sensor 8 and the second finger sensor 9 are recessed into the concave surface 4 of the lower bowl-shaped body 2. Complimentary, the first finger-receiving cavity 10 and the second finger-receiving cavity 11 are positioned opposite to each other across a central cavity 6 of the upper bowl-shaped body 1. The first finger-receiving cavity 10 traverses through the upper bowl-shaped body 1, aligned with the first finger sensor 8. Similarly, the second finger-receiving cavity 11 traverses through the upper bowl-shaped body 1, aligned with the second finger sensor 9. Additionally, the first finger-receiving cavity 10 and the second finger-receiving cavity 11 are shaped identical to the first finger sensor 8 and the second finger sensor 9, respectively. This allows the user to access the BMI reader 7 through the upper bowl-shaped body 1. The recommended timing for measuring one's BMI is prior to eating in order to provide the user with valuable information regarding his or her health.

The space-occupying protrusion 20 limits the amount of food that the present invention may hold, therefore reducing the portion size of the user's meals. The space-occupying protrusion 20 is centrally positioned within the central cavity 6 of the upper bowl-shaped body 1 and is adjacently connected to the concave surface 4 of the upper bowl-shaped body 1. This creates a donut-shaped area within the upper bowl-shaped body 1 in which food is placed. The preferred shape of the space-occupying protrusion 20 is a heart. The heart shape acts as a visual awareness for the user regarding the user's eating habits. Additionally, the space-occupying protrusion 20 may contain a logo embedded on the top surface of the space-occupying protrusion 20. The size, shape, placement, and material composition of the space-occupying protrusion 20 may vary in order to meet the needs and preferences of the user.

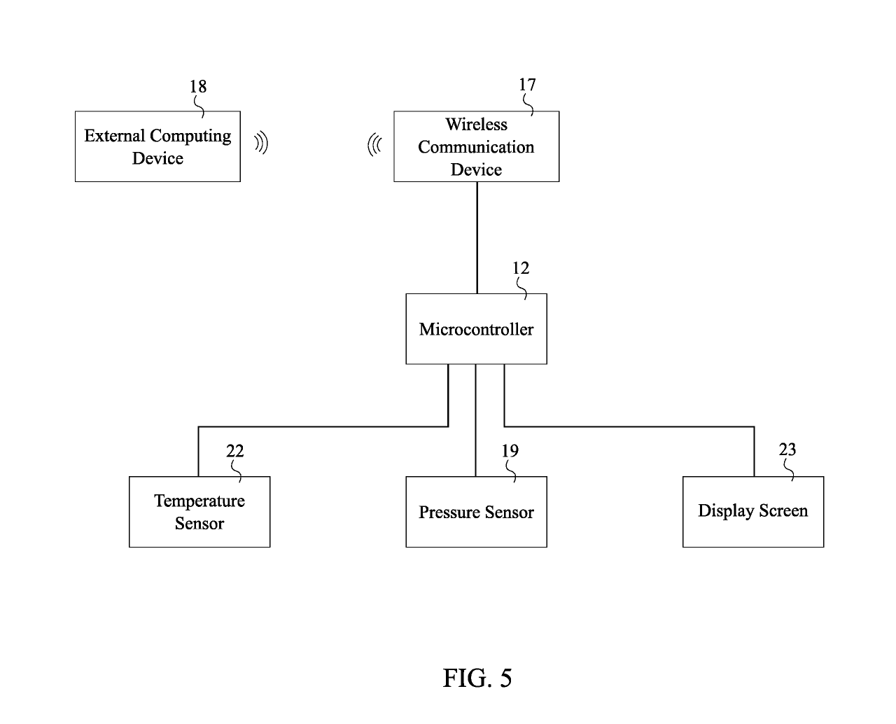

The pressure sensor 19 and the temperature sensor 22 measure the weight of the food within the upper bowl-shaped body 1, the temperature of said food, and the speed with which the food is consumed. This information is then conveyed to the user as feedback about his or her eating habits. The pressure sensor 19 is adjacently connected to the concave surface 4 of the lower bowl-shaped body 2. This ensures that pressure sensor 19 comes in direct contact with the upper bowl-shaped body 1. In particular, when the present invention is in use, the convex surface 5 of the upper bowl-shaped body 1 presses against the pressure sensor 19. This allows the pressure sensor 19 to measure the weight of the food within the upper bowl-shaped body 1. Furthermore, incremental weight measurements over a preset amount of time are then used to calculate the eating speed of the user. A multitude of pressure sensors 19 may be used in order to ensure accurate measurements. The temperature sensor 22 measures the temperature of the food within the upper bowl-shaped body 1. In particular, the temperature sensor 22 is positioned within the central cavity 6 of the lower bowl-shaped body 2 and is mechanically integrated into the concave surface 4 of the lower bowl-shaped body 2. A variety of different devices may be used as the temperature sensor 22. The information gathered by the pressure sensor 19 and the temperature sensor 22 are received and managed by the microcontroller 12. For this, the temperature sensor 22 and the pressure sensor 19 are each electronically connected to the microcontroller 12 as seen in FIG. 5.

Referring to FIG. 5, the aforementioned gathered data is conveyed to the user through the wireless connection to the external computing device 18. The wireless communication device 17 allows for data to be transferred wirelessly. The wireless communication device 17 is preferably internally mounted within the housing base 13 and is electronically connected to the microcontroller 12. The external computing device 18 is any computing device owned by the user. Types of devices that may be used as the external computing device 18 includes, but is not limited to, smart phones, laptops, tablets, and other portable or non-portable computing devices. The wireless communication device 17 is communicably coupled to the external computing device 18 in order to allow for the flow of data in between the microcontroller 12 and the external computing device 18. The present invention may additionally include a companion software application that may be run on the external computing device 18. The companion software application receives and manages the data received from the microcontroller 12 and display said information to the user. Through the companion software application, the user is able to log in and view various data relating to his or her eating habits. For example, the companion software application can record the user's BMI each time the readings are taken to display a chart showing trends to raise awareness of the user's health and weight. Further, eating times can be recorded to display whether or not the user should slow down to prevent over-eating.

Referring to FIG. 2, in the preferred embodiment, the present invention also comprises a display screen 23 and a screen-viewing cavity 24. The display screen 23 directly presents temperature data to the user. The display screen 23 is preferably relatively small and rectangular. The display screen 23 is positioned adjacent to the central cavity 6 of the lower bowl-shaped body 2 and is mechanically integrated into the concave surface 4 of the lower bowl-shaped body 2. This ensures that the user's field of view of the display screen 23 is not obstructed by food items within the upper bowl-shaped body 1. The display screen 23 is electronically connected to the microcontroller 12 and displays information and data received from the microcontroller 12. The main information displayed by the display screen 23 is temperature measurements obtained from the temperature sensor 22. The screen-viewing cavity 24 traverses through the upper bowl-shaped body 1 and reveals the display screen 23 below. More specifically, the screen-viewing cavity 24 is shaped and sized complimentary to the display screen 23 and is aligned with the display screen 23.

The lower bowl-shaped body 2 is not dishware safe and needs to be removed from the upper bowl-shaped body 1 before cleaning because the lower bowl-shaped body 2 houses and supports the electrical components of the present invention. As mentioned before, the upper bowl-shaped body 1 is attached to the lower bowl-shaped body 2. This allows the user to remove the upper bowl-shaped body 1, after use, for cleaning purposes since the upper bowl-shaped body 1 holds the food. A variety of means may be used to attach the upper bowl-shaped body 1 and the lower bowl-shaped body 2. In the preferred embodiment, the upper bowl-shaped body 1 is threadably engaged to the lower bowl-shaped body 2 for easy attachment and detachment. More specifically, the upper bowl-shaped body 1 and the lower bowl-shaped body 2 are attached to each other by a first annular lip 25 and a second annular lip 26. Referring to FIG. 4, the first annular lip 25 is positioned about a rim 3 of the upper bowl-shaped body 1. Additionally, the first annular lip 25 is adjacently connected to the convex surface 5 of the upper bowl-shaped body 1. Similarly, the second annular lip 26 is positioned about a rim 3 of the lower bowl-shaped body 2 and is adjacently connected to the concave surface 4 of the lower bowl-shaped body 2. When the upper bowl-shaped body 1 is attached to the lower bowl-shaped body 2, the first annular lip 25 is threadably attached to the second annular lip 26.

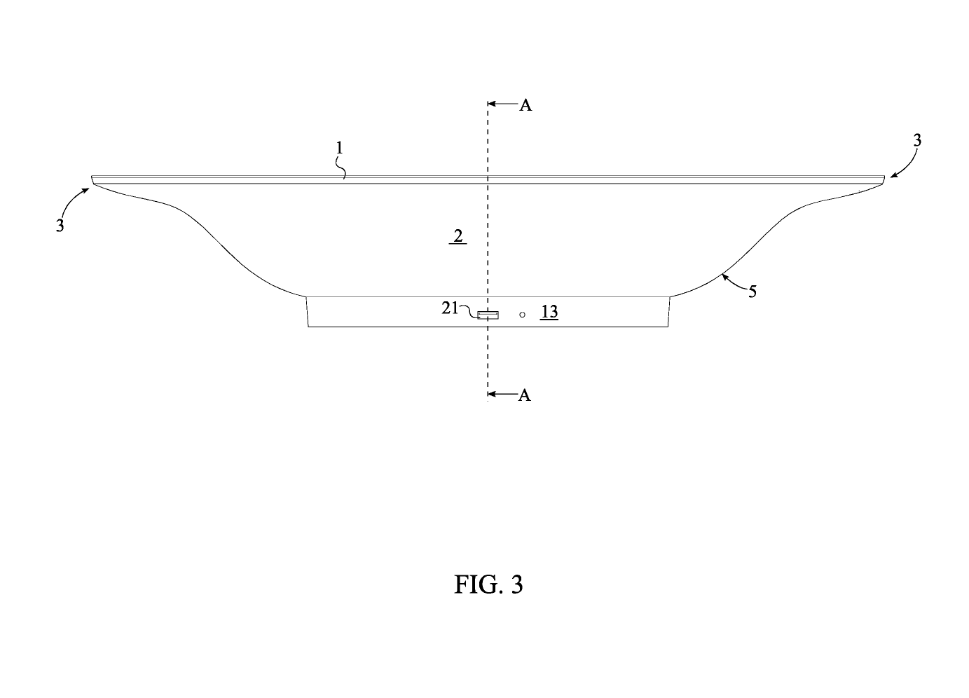

It is preferred that the first annular lip 25 and the second annular lip 26 are shaped such that, when attached, the upper bowl-shaped body 1 is flush with the lower bowl-shaped body 2 with no gaps or breaks. This ensures no food particles may enter the space in between the upper bowl-shaped body 1 and the lower bowl-shaped body 2. A lip-receiving cavity may be utilized to further ensure a flush junction between the upper bowl-shaped body 1 and the lower bowl-shaped body 2 as seen in FIG. 3. The lip-receiving cavity is positioned about the first annular lip 25 and traverses into the upper bowl-shaped body 1, parallel to the first annular lip 25. In order to remove the upper bowl-shaped body 1, the user simply rotates the upper bowl-shaped body 1 counterclockwise relative to the lower bowl-shaped body 2 until detachment. In alternative embodiments of the present invention, alternative interlocking methods and means may be used to attach the upper bowl-shaped body 1 and the lower bowl-shaped body 2. Such means include, but are not limited to, interlocking snaps, buttons, living hinge, and other similar methods.

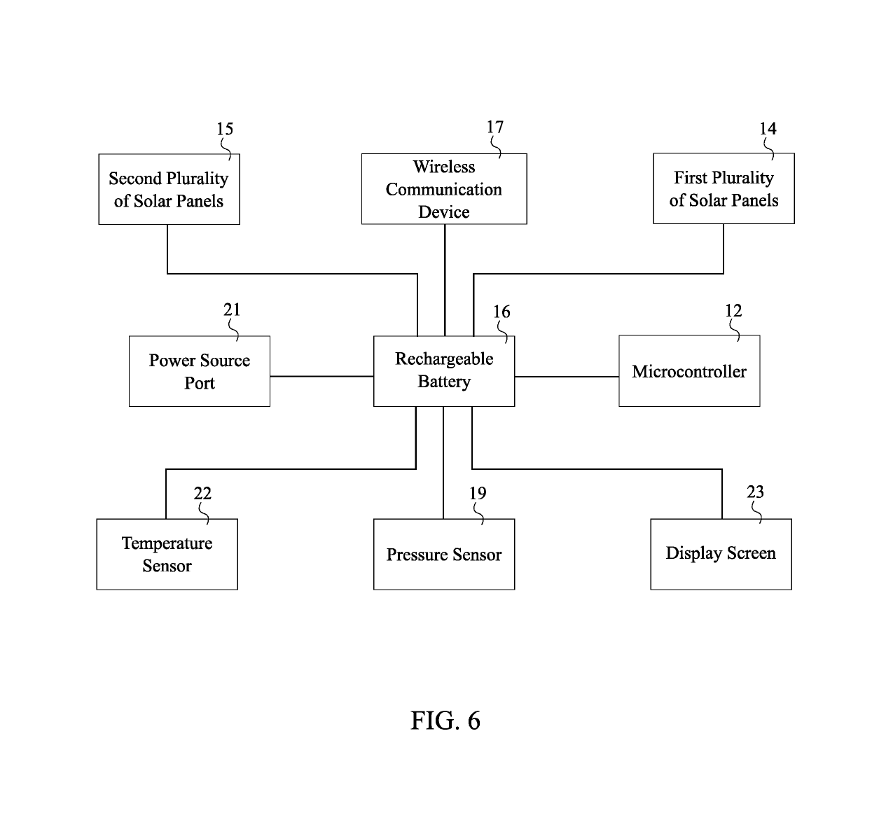

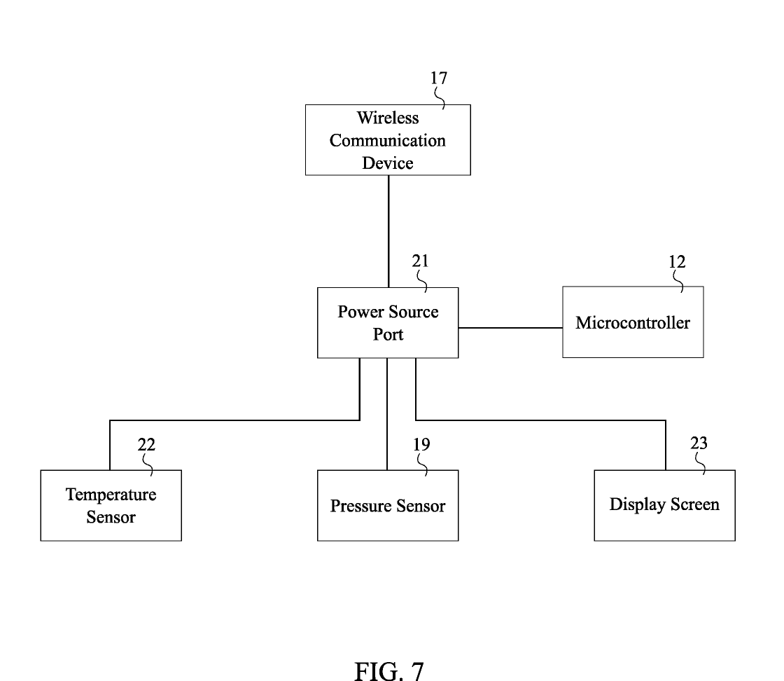

Referring to FIG. 6 and FIG. 7, the electronic components of the present invention may be powered by a direct wired connection to an outlet or a rechargeable battery 16. In one embodiment, the present invention is wired. More specifically, the present invention comprises a power source port 21 that allows an external power source to connect to the electrical components of the present invention. The power source port 21 is laterally integrated into the housing base 13. Furthermore, the power source port 21 is electrically connected to the microcontroller 12, the BMI reader 7, the temperature sensor 22, the display screen 23, the pressure sensor 19, the temperature sensor 22, the wireless communication device 17, and any other electrical component utilized by the present invention. Type of ports that may be used as the power source port 21 include, but are not limited to, universal serial bus (USB) ports, lightning ports, micro USB ports, and other similar devices. In an alternative embodiment, the present invention utilizes the rechargeable battery 16. In this embodiment, the rechargeable battery 16 is internally mounted within the housing base 13 and is electrically connected to the microcontroller 12, the BMI reader 7, the temperature sensor 22, the display screen 23, the pressure sensor 19, the temperature sensor 22, the wireless communication device 17, and any other electrical component utilized by the present invention. The rechargeable battery 16 may be detachable, thus allowing the user to remove the rechargeable battery 16 for external recharging purposes. In one embodiment, the power source port 21 is utilized in conjunction with the rechargeable battery 16 in order to charge the rechargeable battery 16 and or power the electrical components of the present invention.

Referring to FIG. 6, in one embodiment, the present invention further utilizes a first plurality of solar panels 14 and a second plurality of solar panels 15 in order to power the electronic components. The first plurality of solar panels 14 is perimetrically distributed about the first finger sensor 8 with each of the first plurality of solar panels 14 being adjacently connected to the concave surface 4 of the lower bowl-shaped body 2. Similarly, the second plurality of solar panels 15 is perimetrically distributed about the second finger sensor 9 with each of the second plurality of solar panels 15 being adjacently connected to the concave surface 4 of the lower bowl-shaped body 2. It is preferred that the first plurality of solar panels 14 and the second plurality of solar panels 15 are sealed to prevent damage due to fluid and debris exposure. In this embodiment, the first finger-receiving sensor and the second finger-receiving cavity 11 are sized to accommodate the first plurality of solar panels 14 and the second plurality of solar panels 15, i.e. the first finger-receiving cavity 10 and the second finger-receiving cavity 11 are enlarged. This exposes the first plurality of solar panels 14 and the second plurality of solar panels 15 to light. In this embodiment, the rechargeable battery 16 is charged by the first plurality of solar panels 14 and the second plurality of solar panels 15. Thus, the rechargeable battery 16 is electrically connected to the first plurality of solar panels 14 and the second plurality of solar panels 15.

Although the invention has been explained in relation to its preferred embodiment, it is to be understood that many other possible modifications and variations can be made without departing from the spirit and scope of the invention as hereinafter claimed.

What is claimed is:

1. A smart dishware with integrated body mass index (BMI) reader comprises:
an upper bowl-shaped body;
a lower bowl-shaped body;
a BMI reader;
a microcontroller;
a housing base;
the BMI reader comprises a first finger sensor and a second finger sensor;
the upper bowl-shaped body and the lower bowl-shaped body each comprise a concave surface and a convex surface;
the first finger sensor and the second finger sensor being mounted to the lower bowl-shaped body;
the concave surface of the lower bowl-shaped body being pressed against the convex surface of the upper bowl-shaped body;
the lower bowl-shaped body being attached to the upper bowl-shaped body;
the housing base being positioned adjacent to the lower bowl-shaped body, opposite the upper bowl-shaped body;
the housing base being centrally connected to the lower bowl-shaped body;
the microcontroller being internally mounted within the housing base; and
the microcontroller being electronically connected to the BMI reader.

2. The smart dishware with an integrated BMI reader as claimed in claim 1 comprises:
a first finger-receiving cavity;
a second finger-receiving cavity;
the first finger sensor and the second finger sensor being positioned opposite to each other across a central cavity of the lower bowl-shaped body;
the first finger sensor and the second finger sensor each being integrated into the concave surface of the lower bowl-shaped body;
the first finger-receiving cavity and the second finger-receiving cavity being positioned opposite to each other across a central cavity of the upper bowl-shaped body;
the first finger-receiving cavity traversing through the upper bowl-shaped body;
the first finger-receiving cavity being aligned with the first finger sensor;
the second finger-receiving cavity traversing through the upper bowl-shaped body; and
the second finger-receiving cavity being aligned with the second finger sensor.

3. The smart dishware with an integrated BMI reader as claimed in claim 1 comprises:
a first plurality of solar panels;
a second plurality of solar panels;
a rechargeable battery;
the first plurality of solar panels being perimetrically distributed about the first finger sensor;
each of the first plurality of solar panels being adjacently connected the concave surface of the lower bowl-shaped body;
the second plurality of solar panels being perimetrically distributed about the second finger sensor;
each of the second plurality of solar panels adjacently connected the concave surface of the lower bowl-shaped body;
the rechargeable battery being internally mounted within housing base; and
the rechargeable battery being electrically connected to the microcontroller, the first plurality of solar panels, the second plurality of solar panels, and the BMI reader.

4. The smart dishware with an integrated BMI reader as claimed in claim 1 comprises:
a wireless communication device;
an external computing device;
the wireless communication device being internally mounted within the housing base;
the wireless communication device being electronically connected to the microcontroller; and
the wireless communication device being communicably coupled to the external computing device.

5. The smart dishware with an integrated BMI reader as claimed in claim 1 comprises:
an at least one pressure sensor;
the pressure sensor being electronically connected to the microcontroller;
the pressure sensor being adjacently connected to the concave surface of the lower bowl-shaped body; and
the convex surface of the upper bowl-shaped body pressing against the pressure sensor.

6. The smart dishware with an integrated BMI reader as claimed in claim 1 comprises:
a space-occupying protrusion;
the space-occupying protrusion being centrally positioned within the central cavity of the upper bowl-shaped body; and
the space-occupying protrusion being adjacently connected to the concave surface of the upper bowl-shaped body.

7. The smart dishware with an integrated BMI reader as claimed in claim 1 comprises:
a first annular lip;
a second annular lip;
the first annular lip being positioned about a rim of the upper bowl-shaped body;
the first annular lip being adjacently connected to the convex surface of the upper bowl-shaped body;
the second annular lip being positioned about a rim of the lower bowl-shaped body;
the second annular lip being adjacently connected to the concave surface of the lower bowl-shaped body; and
the first annular lip being threadably attached to the second annular lip.

8. The smart dishware with an integrated BMI reader as claimed in claim 1 comprises:
a power source port;
the power source port being electrically connected to the microcontroller and the BMI reader; and
the power source port being laterally integrated into the housing base.

9. The smart dishware with an integrated BMI reader as claimed in claim 1 comprises:
a temperature sensor;
the temperature sensor being positioned within the central cavity of the lower bowl-shaped body;
the temperature sensor being mechanically integrated into the concave surface of the lower bowl-shaped body; and
the temperature sensor being electronically connected to the microcontroller.

10. The smart dishware with an integrated BMI reader as claimed in claim 1 comprises:
a display screen;
a screen-viewing cavity;
the display screen being positioned adjacent to the central cavity of the lower bowl-shaped body;
the display screen being mechanically integrated into the concave surface of the lower bowl-shaped body;
the screen-viewing cavity traversing through the upper bowl-shaped body;
the screen-viewing cavity being aligned with the display screen; and
the microcontroller being electronically connected to the temperature sensor and the display screen.

11. A smart dishware with integrated body mass index (BMI) reader comprises:
an upper bowl-shaped body;
a lower bowl-shaped body;
a BMI reader;
a microcontroller;
a housing base;
a space-occupying protrusion;
the BMI reader comprises a first finger sensor and a second finger sensor;
the upper bowl-shaped body and the lower bowl-shaped body each comprise a concave surface and a convex surface;
the first finger sensor and the second finger sensor being mounted to the lower bowl-shaped body;
the concave surface of the lower bowl-shaped body being pressed against the convex surface of the upper bowl-shaped body;
the lower bowl-shaped body being attached to the upper bowl-shaped body;
the housing base being positioned adjacent to the lower bowl-shaped body, opposite the upper bowl-shaped body;
the housing base being centrally connected to the lower bowl-shaped body;
the microcontroller being internally mounted within the housing base;
the microcontroller being electronically connected to the BMI reader;
the space-occupying protrusion being centrally positioned within the central cavity of the upper bowl-shaped body; and
the space-occupying protrusion being adjacently connected to the concave surface of the upper bowl-shaped body.

12. The smart dishware with an integrated BMI reader as claimed in claim 11 comprises:
a first finger-receiving cavity;
a second finger-receiving cavity;
the first finger sensor and the second finger sensor being positioned opposite to each other across a central cavity of the lower bowl-shaped body;
the first finger sensor and the second finger sensor each being integrated into the concave surface of the lower bowl-shaped body;
the first finger-receiving cavity and the second finger-receiving cavity being positioned opposite to each other across a central cavity of the upper bowl-shaped body;
the first finger-receiving cavity traversing through the upper bowl-shaped body;
the first finger-receiving cavity being aligned with the first finger sensor;
the second finger-receiving cavity traversing through the upper bowl-shaped body; and
the second finger-receiving cavity being aligned with the second finger sensor.

13. The smart dishware with an integrated BMI reader as claimed in claim 11 comprises:
a first plurality of solar panels;
a second plurality of solar panels;
a rechargeable battery;
the first plurality of solar panels being perimetrically distributed about the first finger sensor;
each of the first plurality of solar panels being adjacently connected the concave surface of the lower bowl-shaped body;
the second plurality of solar panels being perimetrically distributed about the second finger sensor;
each of the second plurality of solar panels adjacently connected the concave surface of the lower bowl-shaped body;
the rechargeable battery being internally mounted within housing base; and
the rechargeable battery being electrically connected to the microcontroller, the first plurality of solar panels, the second plurality of solar panels, and the BMI reader.

14. The smart dishware with an integrated BMI reader as claimed in claim 11 comprises:
a wireless communication device;
an external computing device;
the wireless communication device being internally mounted within the housing base;
the wireless communication device being electronically connected to the microcontroller; and
the wireless communication device being communicably coupled to the external computing device.

15. The smart dishware with an integrated BMI reader as claimed in claim 11 comprises:
an at least one pressure sensor;
the pressure sensor being electronically connected to the microcontroller;
the pressure sensor being adjacently connected to the concave surface of the lower bowl-shaped body; and
the convex surface of the upper bowl-shaped body pressing against the pressure sensor.

16. The smart dishware with an integrated BMI reader as claimed in claim 11 comprises:
a first annular lip;
a second annular lip;
the first annular lip being positioned about a rim of the upper bowl-shaped body;
the first annular lip being adjacently connected to the convex surface of the upper bowl-shaped body;
the second annular lip being positioned about a rim of the lower bowl-shaped body;
the second annular lip being adjacently connected to the concave surface of the lower bowl-shaped body; and
the first annular lip being threadably attached to the second annular lip.

17. The smart dishware with an integrated BMI reader as claimed in claim 1 comprises:
a power source port;
the power source port being electrically connected to the microcontroller and the BMI reader; and
the power source port being laterally integrated into the housing base.

18. The smart dishware with an integrated BMI reader as claimed in claim 11 comprises:
a temperature sensor;
the temperature sensor being positioned within the central cavity of the lower bowl-shaped body;
the temperature sensor being mechanically integrated into the concave surface of the lower bowl-shaped body; and
the temperature sensor being electronically connected to the microcontroller.

19. The smart dishware with an integrated BMI reader as claimed in claim 11 comprises:
a display screen;
a screen-viewing cavity;
the display screen being positioned adjacent to the central cavity of the lower bowl-shaped body;
the display screen being mechanically integrated into the concave surface of the lower bowl-shaped body;
the screen-viewing cavity traversing through the upper bowl-shaped body;
the screen-viewing cavity being aligned with the display screen; and
the microcontroller being electronically connected to the temperature sensor and the display screen.
